(19) United States
(12) Patent Application Publication
(10) Pub. No.: US 2020/0197681 A1
(43) Pub. Date: Jun. 25, 2020

(54) Implantable Unique Device Identifier And Detection System

(71) Applicant: Bard Peripheral Vascular, Inc., Tempe, AZ (US)

(72) Inventors: Kyle Bodnicki, Phoenix, AZ (US); Andrzej J. Chanduszko, Chandler, AZ (US); Long Chen, Tucson, AZ (US); Kim R. Ligouri, Tempe, AZ (US); Lenny Lopez, Tucson, AZ (US); Genevieve Messina, Tucson, AZ (US); Peng Zheng, Chandler, AZ (US)

(21) Appl. No.: 16/608,767

(22) PCT Filed: Apr. 20, 2018
(86) PCT No.: PCT/US2018/028605

§ 371 Date: Oct. 25, 2019

Related U.S. Application Data
(60) Provisional application No. 62/491,846, filed on Apr. 28, 2017.

(51) Int. Cl. A61M 39/02

(52) U.S. Cl. CPC A61M 39/0208; A61M 25/0097; A61M 2039/0238

(57) ABSTRACT

Disclosed is an implantable medical device such as a port assembly including a catheter lock and one or more unique device identifiers (“UDIs”) of the catheter lock. The catheter lock is configured to fit over an end portion of a catheter over an outlet stem extending from a portion of the implantable medical device such as housing of a port. The one or more UDIs embedded in the catheter lock include machine-readable identification data for the implantable medical device. Also disclosed is a system including the implantable medical device and instructions stored in a memory of a computing device for execution by one or more processors. Methods related to the foregoing are additionally disclosed.

### PRIORITY

This application claims the benefit of priority to U.S. Provisional Patent Application No. 62/491,846, filed Apr. 28, 2017, titled “Implantable Unique Device Identifier and Detection System,” which is hereby incorporated herein by reference in its entirety.

### BACKGROUND

Implantable ports, or simply “ports,” such as central venous access ports provide a convenient method to repeatedly deliver a substance to remote areas of the body by way of an attached catheter without utilizing surgical procedures each time. Ports are implantable within the body (e.g., subcutaneously) and permit the infusion of medicine, parenteral solutions, blood products, or other fluids. Additionally, ports are also used for blood sampling. In common practice, a port is implanted within the body, and a catheter is connected to the port in fluid communication therewith. The catheter is routed to a remote area where a fluid is desired to be delivered or removed. To deliver the fluid, a caregiver locates a septum of the port by palpation of a patient's skin. Port access is accomplished by percutaneously inserting a needle, typically a non-coring needle, through the septum of the port and into a reservoir of the port. A fluid containing a drug or some other beneficial substance can then be administered by bolus injection or continuous infusion into the reservoir of the port. The fluid then flows through the reservoir into the catheter and finally to the remote site where the fluid is desired.

One particular type of port is a power injectable port. Power injectable ports are structured for use in computed tomography (“CT”) scanning processes, where a power injector system is employed for injecting contrast media through the power injectable port into a peripherally inserted intravenous (“IV”) line. Various power injectable ports, assemblies, and systems are disclosed in the following patents: U.S. Pat. Nos. 9,682,186; 9,603,993; 9,603,992; 9,474,888; 8,998,860; 8,939,947; 8,603,052; 8,585,663; 8,382,724; 8,382,723; 8,202,259; 8,029,482; 7,959,615; 7,947,022; 7,785,302; 8,805,478; 8,641,688; 8,545,460; 8,475,417; 8,025,639; 8,608,713; 8,177,762, each of which is hereby incorporated herein in its entirety in this application.

Power injectable ports can be difficult to identify once implanted in a human body; however, identification is necessary to ensure that an implanted port is properly structured for use in a CT scanning process. Identification of such ports or other implanted medical devices can be important for a variety of other reasons as well. Accordingly, there is a need to facilitate identification of medical devices such as ports or assemblies including such ports once such medical devices are implanted. The patents set forth above disclose various means to identify an implanted power injectable port including, for example, structural features of the port, palpable septum protrusions or bumps, radiopaque identifying features of the port observable via imaging technology such as X-ray, and combinations thereof. Notwithstanding the foregoing means for identification, identification of implanted medical devices such as implanted ports or assemblies including such ports is of ongoing importance.

Disclosed herein are various embodiments of systems, devices, and methods thereof that facilitate the identification of an implanted medical device.

### SUMMARY

Disclosed herein is an implantable medical device including, in some embodiments, a catheter lock and one or more unique device identifiers (“UDIs”) embedded in the catheter lock. The catheter lock is configured to fit over an end portion of a catheter over a nipple of an outlet stem extending from a housing. The one or more UDIs embedded in the catheter lock include machine-readable identification data for the implantable medical device.

In some embodiments, the identification data for the implantable medical device is identification data for a port assembly. The housing is that of a port of the port assembly, which includes a needle-penetrable septum that defines a top of a reservoir disposed within the housing of the port. The catheter is configured for accessing at least a vein of a patient, the catheter having a lumen in fluid communication with an outlet in the housing of the port.

In some embodiments, two or more UDIs are embedded in the catheter lock approximately equally spaced around the catheter lock. Each UDI of the two or more UDIs includes the same identification data for the implantable medical device, thereby facilitating machine reading of the UDIs.

In some embodiments, each UDI is an identification tag selected from a radio-frequency identification (“RFID”) tag and a near-field communication (“NFC”) tag.

In some embodiments, each UDI is a RFID tag.

In some embodiments, each UDI is a passive RFID tag.

Also disclosed herein is a system including, in some embodiments, an implantable medical device and instructions stored in a memory of a computing device for execution by one or more processors of the computing device configured to cause the computing device to present identification data for the implantable medical device to a user on a display screen associated with the computing device. The implantable medical device includes a catheter lock and one or more UDIs embedded in or coupled to the catheter lock. The catheter lock is configured to fit over an end portion of a catheter over an outlet stem extending from a housing. The one or more UDIs embedded in or coupled to the catheter lock include machine-readable identification data for the implantable medical device.

In some embodiments, the implantable medical device includes a port assembly. The identification data for the implantable medical device includes identification data for the port assembly selected from maker of a port of the port assembly, model of the port, lot number for a lot of the port, serial number of the port, magnetic resonance imaging (“MRI”) safety information for the port or the port assembly, and a description of the port assembly.

In some embodiments, the port includes a power injectable port. The housing is that of the power injectable port configured for mechanically assisted pressurized injections to achieve a desired flow rate of injectant through the port assembly. The housing includes a needle-penetrable septum that defines a top of a reservoir disposed within the housing of the power injectable port. The catheter is configured for accessing at least a vein of a patient, the catheter having a lumen in fluid communication with an outlet in the housing of the power injectable port.

In some embodiments, each UDI is an identification tag selected from an RFID tag and an NFC tag.

In some embodiments, each UDI is an RFID tag.

In some embodiments, each UDI is an NFC tag.

In some embodiments, the instructions are further configured to cause the computing device to accept user input through a user-input mechanism of the computing device for updating or overwriting the identification data for the implantable medical device in each UDI.

In some embodiments, the system further includes a dedicated UDI reader including memory storing instructions for execution by one or more processors of the UDI reader configured to cause the UDI reader to read the identification data for the implantable medical device and optionally update or overwrite the identification data for the implantable medical device in each UDI.

In some embodiments, the computing device and the UDI reader are each further configured via respective instructions thereof to communicate the identification data for the implantable medical device to the other through a short-range wireless-communication interface.

In some embodiments, the instructions are configured for a computing device selected from a mobile computing device and a wearable computing device. The mobile computing device includes a smartphone, a tablet computer, or a dedicated system device. The wearable computing device includes a smartwatch or an optical head-mounted display.

Also disclosed herein is a non-transitory computer-readable medium including instructions for execution by one or more processors of a computing device configured to cause the computing device to perform operations including, in some embodiments, presenting identification data to a user in one or more graphical user interfaces (“GUIs”) on a display screen associated with the computing device read from one or more UDIs of a port assembly. The port assembly includes a catheter lock with the one or more UDIs embedded in or coupled to the catheter lock. The catheter lock is configured to fit over an end portion of a catheter over an outlet stem extending from a housing of a port of the port assembly. Each UDI of the one or more UDIs is an identification tag selected from an RFID tag and an NFC tag embedded in the catheter lock.

In some embodiments, the instructions are further configured to cause the computing device to accept user input through a user-input mechanism of the computing device for updating or overwriting the identification data for the port assembly in each UDI.

In some embodiments, each UDI of the one or more UDIs is an RFID tag, and the instructions are further configured to cause the computing device to cooperate with an RFID tag reader over a short-range wireless-communication interface of the computing device for communications regarding the identification data.

In some embodiments, the port is a power injectable port configured for mechanically assisted pressurized injections to achieve a desired flow rate of injectant through the port assembly. The housing includes a needle-penetrable septum that defines a top of a reservoir disposed within the housing of the power injectable port. The catheter is configured for accessing at least a vein of a patient, the catheter having a lumen in fluid communication with an outlet in the housing of the power injectable port.

These and other features of the concepts provided herein can be better understood with reference to the drawings, description, and appended claims.

### DRAWINGS

FIG. 1 provides a schematic illustrating a port assembly implanted in a human body.

FIG. 2A provides a schematic illustrating a catheter lock with an embedded identification tag for an implantable medical device such as a port assembly in accordance with some embodiments.

FIG. 2B provides a schematic illustrating a perspective view of a catheter lock with a recess for embedding an identification tag for an implantable medical device such as a port assembly in accordance with some embodiments.

FIG. 2C provides a schematic illustrating a side view of a catheter lock with a recess for embedding an identification tag for an implantable medical device such as a port assembly in accordance with some embodiments.

FIG. 2D provides a schematic illustrating a top view of a catheter lock with a recess for embedding an identification tag for an implantable medical device such as a port assembly in accordance with some embodiments.

FIG. 2E provides a schematic illustrating an end view of a catheter lock with a recess for embedding an identification tag for an implantable medical device such as a port assembly in accordance with some embodiments.

FIG. 3 provides a schematic illustrating a port assembly including a catheter lock with an embedded identification tag over a catheter over an outlet stem of a port in accordance with some embodiments.

FIG. 4 provides a schematic illustrating a port assembly including a catheter lock with an identification tag coupled thereto over a catheter over an outlet stem of a port via assembly of a port assembly in accordance with some embodiments.

FIG. 5A provides a schematic illustrating identification data in a GUI associated with a computing device read from a UDI embedded in a port or a catheter lock of a port assembly in accordance with some embodiments.

FIG. 5B provides a schematic illustrating identification data in a GUI written to and subsequently read from a UDI embedded in a power injectable port or a catheter lock of a port assembly in accordance with some embodiments.

FIG. 6 provides a schematic illustrating reading identification data from or writing identification data to an identification tag by a computing device through an intermediate identification tag reader in accordance with some embodiments.

FIG. 7 is a schematic illustrating one or more components of a computing device or an identification tag reader in accordance with some embodiments.

FIG. 8 is a reading-range chart providing reading-range data for a reading-range experimental run for an RFID tag embedded in a catheter lock of a port assembly.

FIG. 9 is an example tensile-strength chart providing tensile-strength data for a tensile-strength experiment involving a catheter lock for a port assembly.

FIG. 10 is a time-response chart comparing response times for two different smartphones using time-response experimental data for reading an identification tag embedded in a catheter lock for a port assembly.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “front,” “back,” “top,” “bottom,” “forward,” “reverse,” “clockwise,” “counter clockwise,” “up,” “down,” or other similar terms such as “upper,” “lower,” “aft,” “fore,” “vertical,” “horizontal,” “proximal,” “distal,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

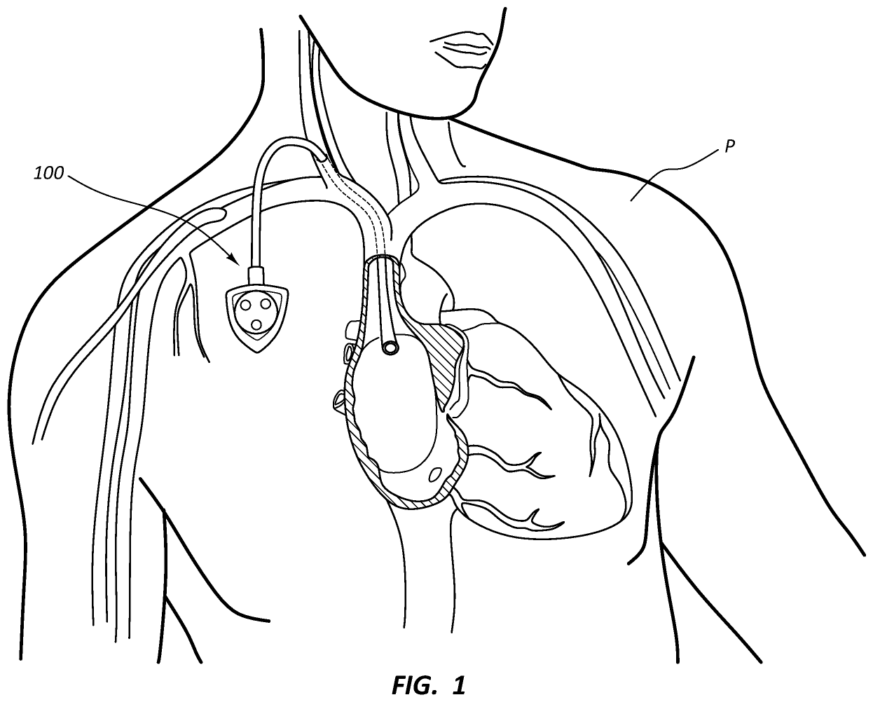

Implanted ports can be used to deliver injections by way of respectively attached catheters. For example, as shown in FIG. 1, a central venous access port combined with a central venous catheter forms a port assembly 100 useful for delivering injections to the superior vena cava of a patient P. Power injectable ports, which are configured for powered injections, are a particular type of implantable port that is structured for use in a CT scanning process. Implantable medical devices including port assemblies with power injectable ports can be difficult to identify once implanted in a human body; however, identification is necessary to ensure that an implanted port is properly structured for use in a CT scanning process. Identification of such ports or other implanted medical devices can be important for a variety of other reasons as well. Accordingly, there is a need to facilitate identification of medical devices such as ports or assemblies including such ports once such medical device are implanted. Various means to identify an implanted power injectable port include, for example, structural features of the port (e.g., a triangular shape), palpable septum protrusions or bumps, radiopaque identifying features of the port observable via imaging technology such as X-ray, and combinations thereof. Notwithstanding the foregoing means for identification, identification of implanted medical devices such as implanted ports or assemblies including such ports are implanted is of ongoing importance.

Disclosed herein are various embodiments of systems, devices, and methods thereof that facilitate the identification of an implanted medical device.

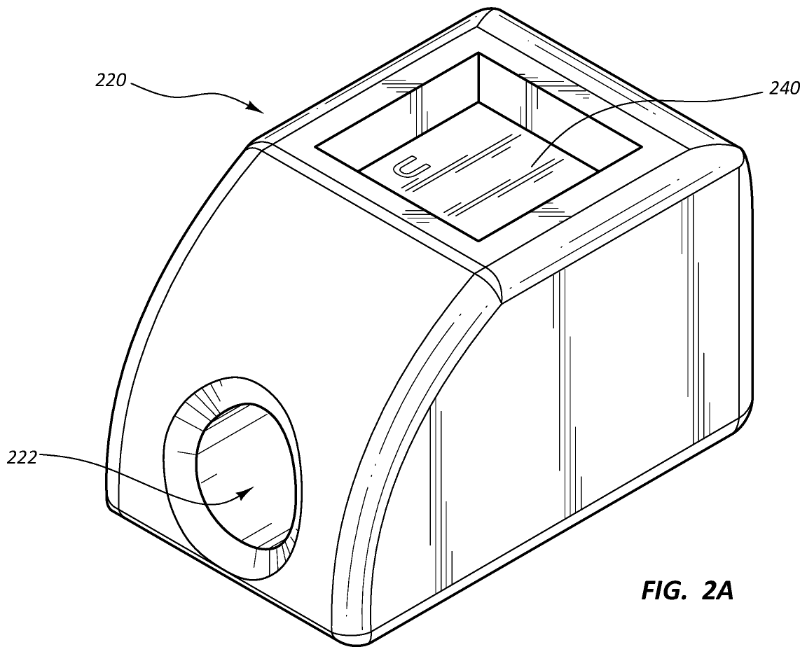

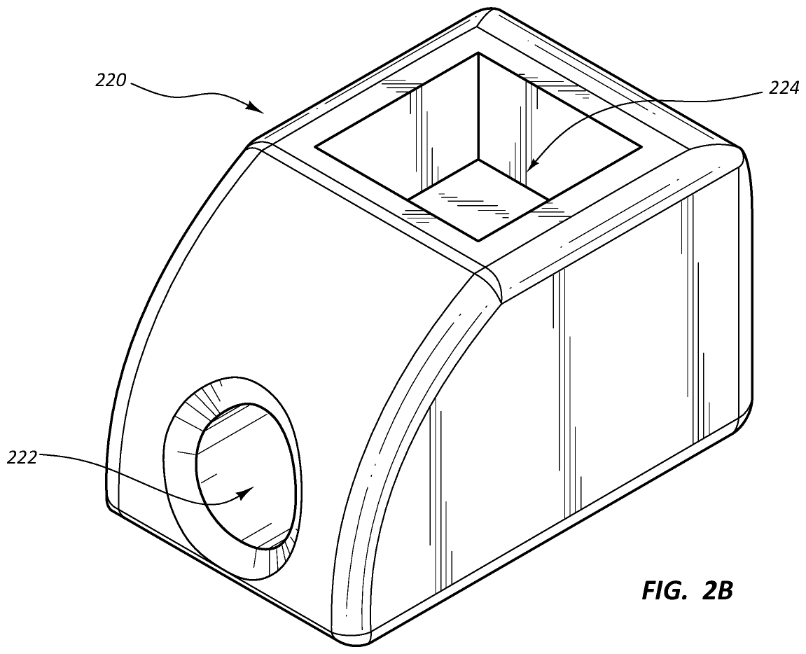

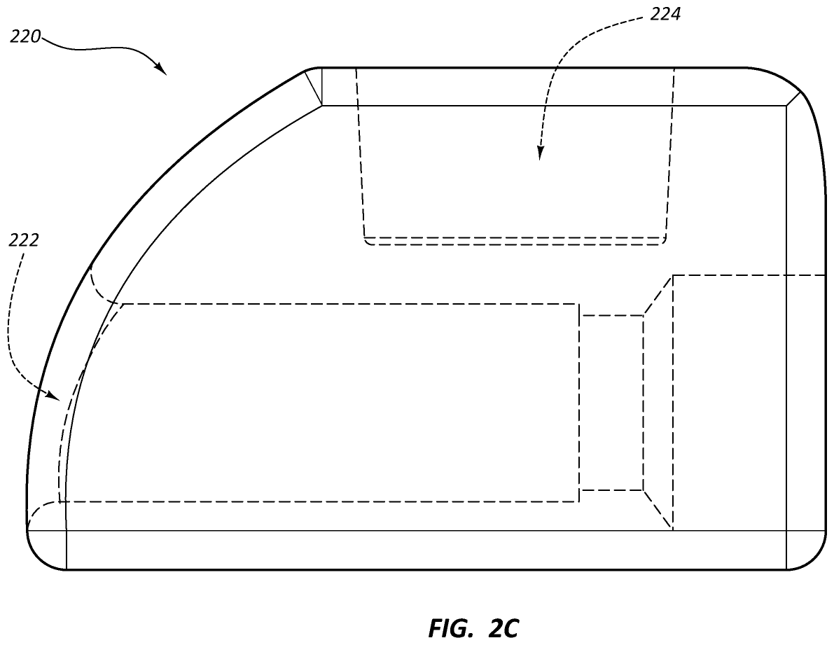

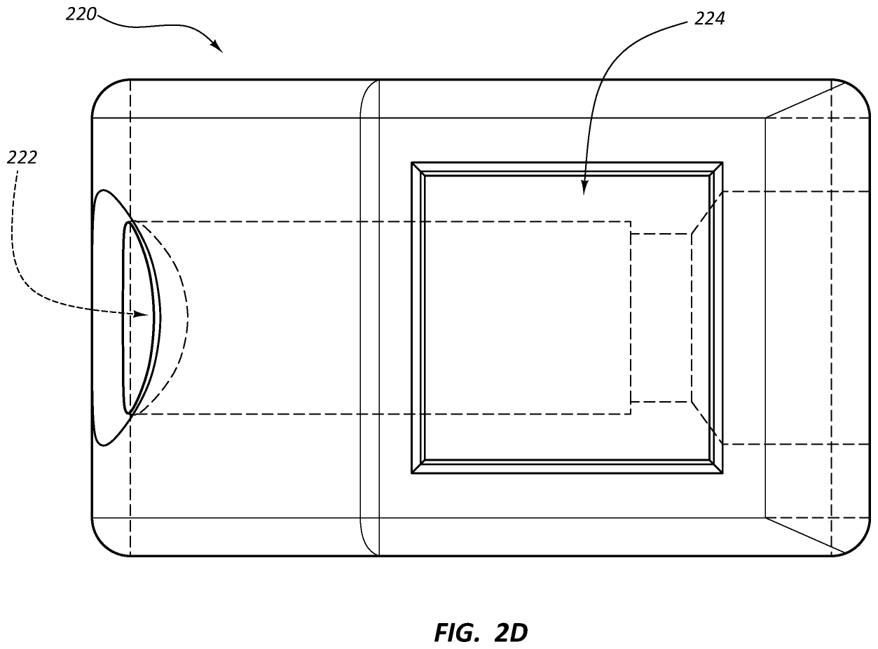

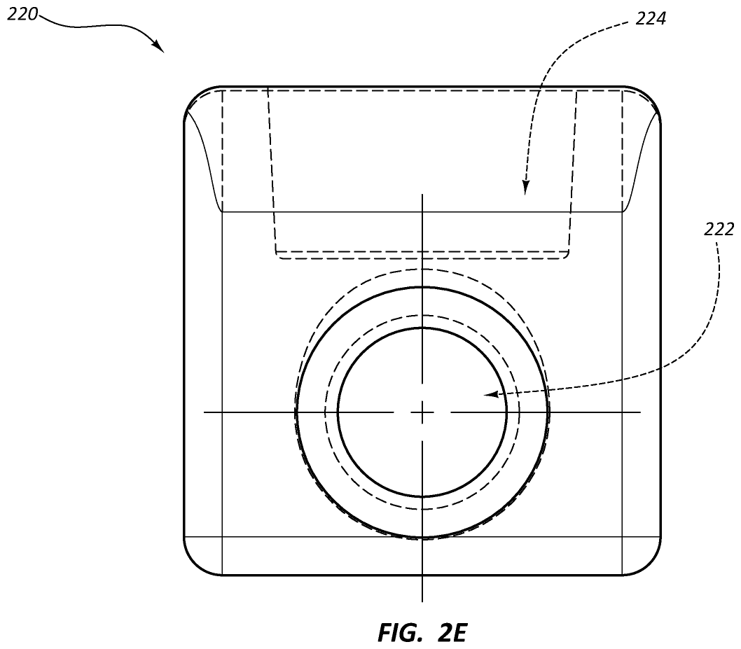

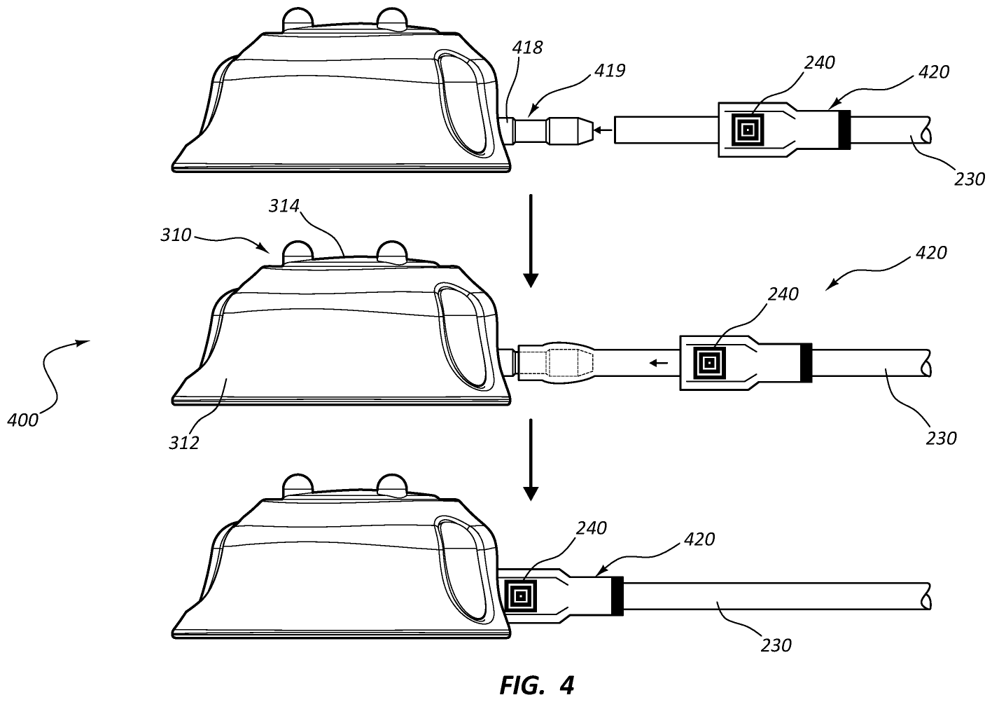

FIG. 2A provides a schematic illustrating a catheter lock 220 with an embedded UDI such as a machine-readable electromagnetic identification tag 240 for an implantable medical device such as the port assembly 100 in accordance with some embodiments. FIGS. 2B-2E provide schematics illustrating various view of the catheter lock 220 with a recess 224 for embedding the identification tag 240. FIG. 4 provides a catheter lock 420 having a different shape than that of the catheter lock 220, but the catheter lock 420 is mentioned here it shares the features disclosed below for catheter lock 220.

The catheter lock 220 can be configured to fit over an end portion of a catheter 230 and lock the catheter 230 on an outlet stem of a port or power injectable port to form a port assembly such as the port assembly 100. The catheter lock 220 can include at least one UDI embedded in the catheter lock 220 or coupled to the catheter lock 220 configured to provide identification data for the port assembly 100. It should be understood that implantable medical devices including such UDIs are not limited to port assemblies or medical devices operable with catheter locks; however, advantageously, existing implantable medical devices operable with catheter locks such as port assemblies can benefit from the catheter locks with the UDIs provided herein without needing to be redesigned.

While a port such as a power injectable port has a larger body than the catheter locks provided herein, and while such ports can more easily accommodate one or more UDIs—particularly larger-sized RFID tags with larger antennae and, hence, greater reading distances—UDIs are counterintuitively embedded in or coupled to catheter locks. But this is because catheter locks can be replaced at less cost than ports should the UDIs be defective at a time of manufacturing or become defective subsequent to manufacturing. Furthermore, a UDI such as an RFID tag on a catheter lock can be more easily oriented (e.g., turned) outward during implantation, which makes it easier for subsequent readings or updates to the UDI, particularly if only one UDI is used with the catheter lock. Moreover, having a UDI embedded in or coupled to a catheter lock serves as a visual reminder during the few last steps of implantation as to the type of catheter (e.g., Groshong line) being locked onto a port or power injectable port.

The catheter lock 220 can include any of a number of UDIs including a single identification tag to many identification tags, same or different, embedded in the catheter lock 220, coupled to the catheter lock 220, or a combination thereof. While the catheter lock 220 of FIGS. 2A-2E shows a single recess 224 for embedding a single identification tag such as the identification tag 240, it should be understood that a number of recesses (e.g., 2, 3, 4, or more recesses) can be included on the catheter lock 220 for embedding more than one identification tag. Likewise, a number of designated areas (e.g., 2, 3, 4, or more areas) can be reserved on the catheter lock 220 for coupling more than one identification tag. The catheter lock 220 can even include a mixture of one or more recesses and one or more designated areas. For example, the catheter lock 220 of FIGS. 2A-2E can include recesses for embedding or designated areas for coupling identification tags on any two opposing sides about a catheter through hole 222 of the catheter lock 220 or on all four sides about the catheter through hole 222. An increase in the number of identification tags—especially machine-readable electromagnetic identification tags of the same type including the same identification data approximately equally spaced around the catheter lock 220—can increase chances that at least one antenna of the number of identification tags is correctly oriented to best receive a polling signal from an identification tag reader.

Any type of machine-readable electromagnetic identification tag of a number of different types of identification tags such as, but not limited to, RFID tags (e.g., read only RFID tags; read-write RFID tags; write once, ready many [“WORM”] RFID tags; and passive RFID tags of the foregoing) and NFC tags can be embedded in or coupled to the catheter lock 220. NFC tags are generally smaller in size than RFID tags, which can be advantageous on small-sized implantable medical devices such as port assemblies. That said, due to the generally larger size of RFID tags, RFID tags have longer antennae and, thus, larger communication ranges, which is also advantageous in certain embodiments. Furthermore, in embodiments of the catheter lock 220 including two or more identification tags, each of the two or more identification tags can be the same or different. Again, an increase in the number of the same type of identification tag (e.g., RFID tag or NFC tag) can increase chances that at least one antenna of the number of identification tags is correctly oriented to best receive a polling signal from an identification tag reader. Having two or more different types of identification tag (e.g., RFID tag and NFC tag) can increase a number of ways by which the identification data can be read from the different identification tags or updated. For example, the catheter lock 220 can include an RFID tag readable by a dedicated RFID tag reader, and the catheter lock can include an NFC tag readable by a smartphone. That said, passive high-frequency RFID tags using ISO 14443 or ISO 15693 can be at least read using an NFC communication interface of a smartphone.

The identification data in an identification tag for an implantable medical device can include identification data selected from maker of the implantable medical device, model of the implantable medical device, lot number for a lot of the implantable medical device, serial number of the implantable medical device, Mill safety information for the implantable medical device, and additional description for the implantable medical device or its implantation depending upon the implantable medical device. For example, the identification data for the identification tag 240 can include identification data for a port assembly such as the port assembly 100, the identification data selected from maker of a port of the port assembly, model of the port (e.g., a central venous access port, a power injectable port, etc.), lot number for a lot of the port, serial number of the port, MRI safety information for the port or the port assembly 100, and additional description for the port assembly 100 such as whether it includes a Groshong line, a Hickman line, or the like. Even further information can be included with the identification data, the further information including, for example, procedural-related information such as date of implantation. An identification tag such as the identification tag 240 can be sized, in terms of memory, in accordance with the amount of information to be stored on the identification tag. In the foregoing example, an identification tag with a minimum of 32 bytes of storage is sufficient to store the identification data. That said, an identification tag in excess of 32 bytes is useful for storing even more identification data if even more additional description is desired.

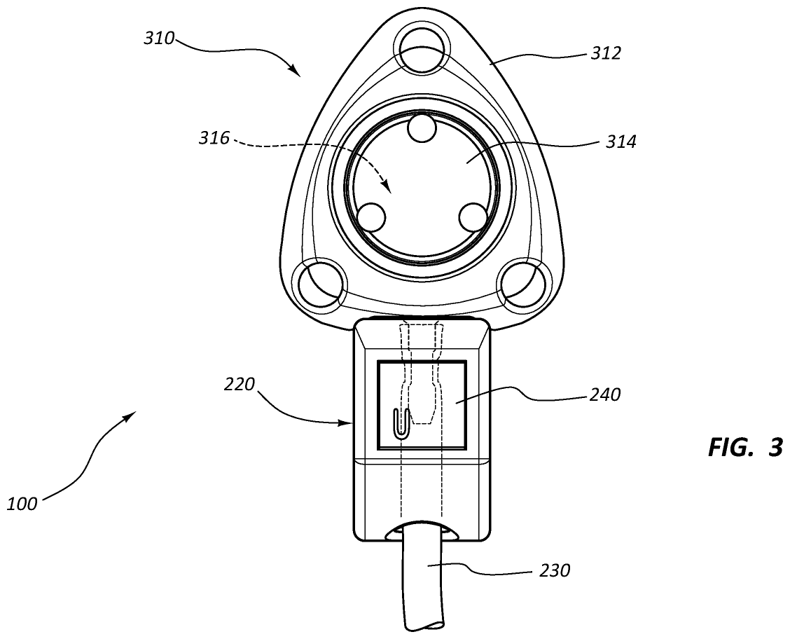

FIG. 3 provides a schematic illustrating the port assembly 100 including the catheter lock 220 with the embedded identification tag 240 over the catheter 230 over an outlet stem of a port 310 in accordance with some embodiments. Likewise, FIG. 4 provides a schematic illustrating port assembly 400 including a catheter lock 420 with the identification tag 240 coupled thereto over the catheter 230 over an outlet stem 418 of the port 310 via assembly of the port assembly 400 in accordance with some embodiments.

As shown, an implantable medical device such as the port assembly 100 or 400 can include a port or power injectable port 310 having a housing 312 (e.g., a non-penetrable housing, a suturable housing of silicone, etc.) with an aperture at a top of the housing 312, an outlet in a side of the housing 312, a self-sealing, needle-penetrable septum 314 (e.g., a silicone septum) over the aperture defining a top of a reservoir disposed within the housing 312, and an outlet stem 418 extending from the housing 312 and fluidly coupled to the outlet in the side of the housing 312. The outlet stem 418 can include a circumferentially recessed portion 419 providing a nipple in an end portion of the outlet stem 418. The port assembly 100 or 400 can further include the catheter 230 (e.g., a radiopaque catheter, a polyurethane catheter, a radiopaque polyurethane catheter, etc.) and a catheter lock such as the catheter lock 220 or 420. The catheter 230, which can be configured for accessing a vein (e.g., superior vena cava) of a patient, can also be configured to fit over the outlet stem 418 including the nipple and at least a portion of the recessed portion 419 of the outlet stem 418, and the catheter lock 220 or 420 can be configured to fit over an end portion of the catheter 230 and a remainder of the outlet stem 418 extending from the housing 312. In such a configuration, the catheter 230 has a lumen in fluid communication with a lumen of the outlet stem 418, which, in turn, is in fluid communication with the reservoir of the port or power injectable port 310. The port assembly 100 or 400 can further include one or more UDIs such as machine-readable electromagnetic identification tags embedded in or coupled to the catheter lock 220 or 420 including identification data for the port assembly 100 or 400.

When the implantable medical device includes the port assembly 100 or 400 with a power injectable port, the power injectable port can be configured for mechanically assisted pressurization to inject, for example, contrast media at a desired flow rate, which is useful in CT scanning processes.

Systems can include one or more implantable medical devices such as any one or more port assemblies and a computing device or at least instructions configured to cause the computing device to cooperate with the one or more implantable medical devices. The computing device can include memory (e.g., a non-transitory computer-readable medium) storing the instructions for execution by one or more processors of the computing device configured to cause the computing device to cooperate with the one or more implantable medical devices. For example, the instructions can be configured to cause the computing device to present identification data for the one or more implantable medical devices to a user on a display screen associated with computing device. Presenting the identification data for the one or more implantable medical devices to the user on the display screen associated with computing device can include presenting the identification data in one or more GUIs on the display screen of the computing device.

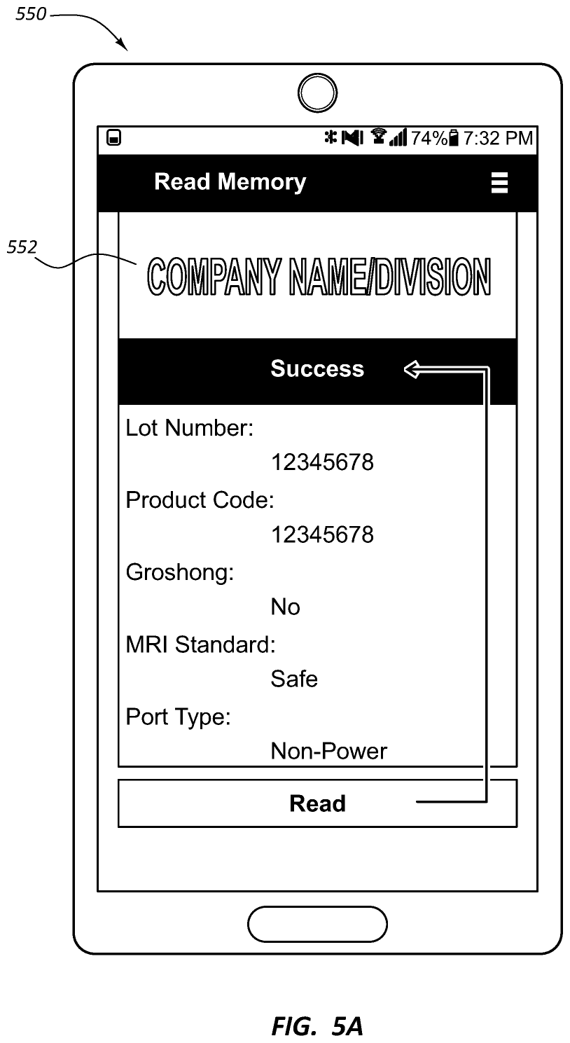

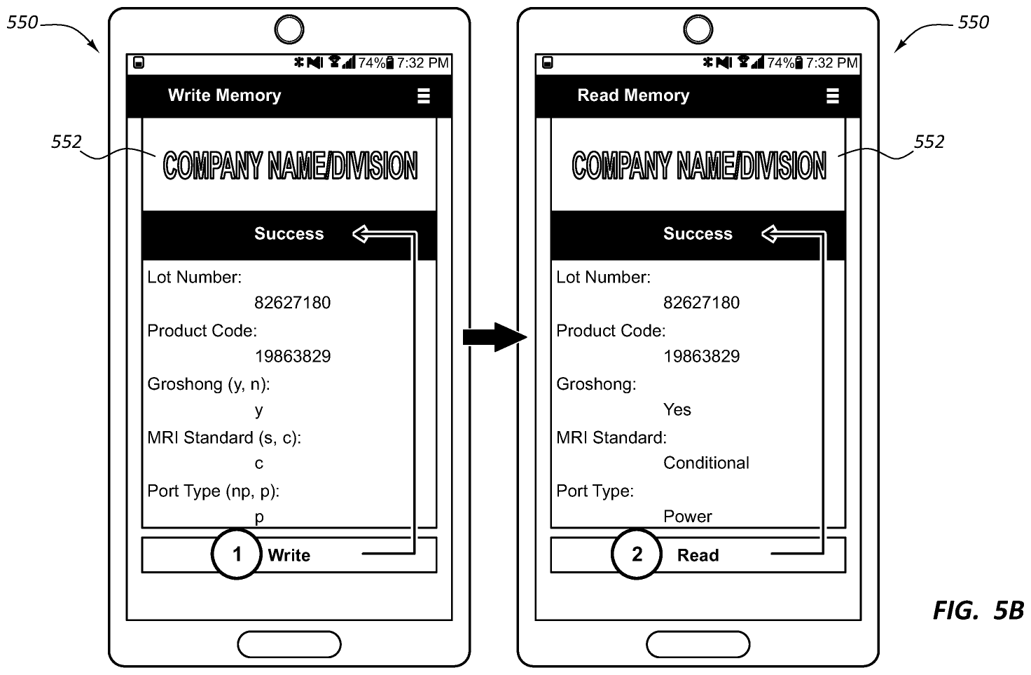

FIG. 5A provides a schematic illustrating identification data in a GUI 552 associated with a computing device 550 read from a UDI such as a machine-readable electromagnetic identification tag embedded in a port or catheter lock of a port assembly in accordance with some embodiments.

As shown, the computing device 550 can be configured to present the identification data in the GUI 552 including a port model (e.g., central venous access port) for the port, a lot number for the port, a serial number or product code for the port, Mill safety information for the port or the port assembly, or additional description for the port or port assembly (e.g., includes a Groshong line)—even procedure-related information such as a procedure date. However, the GUI 552 is not limited to the foregoing data, as the GUI 552 can be configured to accommodate any of a number of fields a UDI such as a machine-readable electromagnetic identification tag can store.

In addition to presenting the identification data in the GUI 552, the instructions for execution by the one or more processors of the computing device 550 can be configured to cause the computing device 550 to accept user input through a user-input mechanism of the computing device 550 for writing, updating, or overwriting the identification data for an implantable medical device such as the foregoing port or power injectable port. This is useful for providing information complementary to the identification data such as procedure-related information, for example, procedure date. The user-input mechanism can include, but is not limited to, a mouse, a touchscreen display screen, and a pen device with character recognition software, each of which can be used with the GUI 552. The user-input mechanism can further include a scanning device (e.g., a smartphone camera) with character recognition software, as well as voice recognition through a voice user interface (“VUI”). The scanning device can also be used to scan a UDI such as a machine-readable optical identification tag, for example, a quick-response (“QR”) code or a universal product code (“UPC”) barcode, on packaging for an implantable medical device to pre-populate one or more fields in the GUI 552 for writing, updating, or overwriting the identification data or other data for the implantable medical device.

FIG. 5B provides a schematic illustrating identification data in the GUI 552 written to and subsequently read from a UDI such as a machine-readable electromagnetic identification tag embedded in a power injectable port or a catheter lock of a port assembly in accordance with some embodiments.

As shown, the computing device 550 can be configured to accept the identification data in the GUI 552 including a port model (e.g., power injectable port) for the port, a lot number for the port, a serial number or product code for the port, Mill safety information for the port assembly, or additional description for the port or port assembly (e.g., includes a Groshong line)—even procedure-related information such as a procedure date. However, again, the GUI 552 is not limited to the foregoing data, as the GUI 552 can be configured to accommodate any of a number of fields a UDI such as a machine-readable electromagnetic identification tag can store.

The computing device 550 can include, but is not limited to, mobile computing devices such as smartphones, tablet computers, and dedicated system devices (e.g., devices designed primarily for reading from or writing to electromagnetic identification tags), as well as wearable computing devices including smartwatches and optical head-mounted displays for augmented reality.

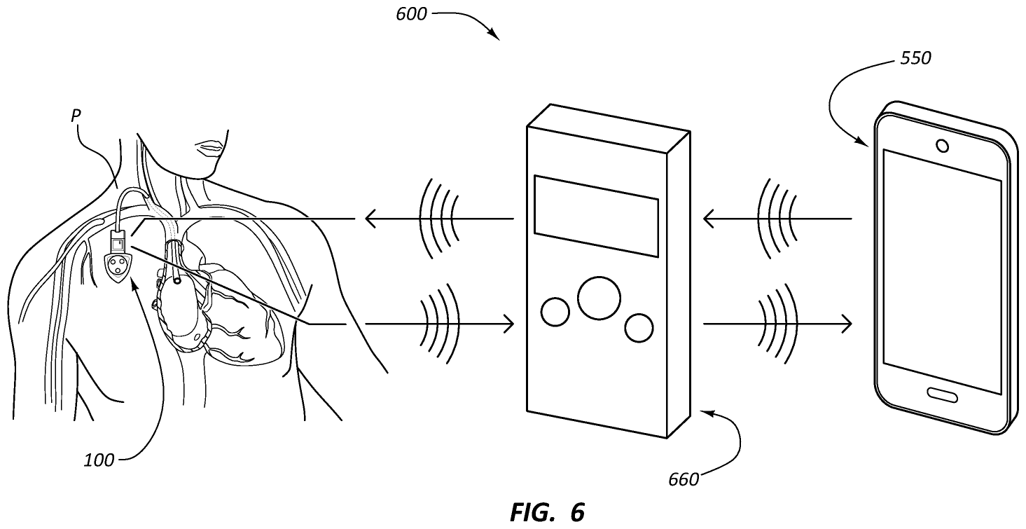

Systems provided herein can also include one or more implantable medical devices (e.g., port assemblies including ports or power injectable ports), a computing device such as the computing device 550 or at least the instructions configured to cause the computing device to cooperate with the one or more implantable medical devices, and an identification tag reader or identification tag reader-writer, hereinafter simply “identification tag reader,” or at least instructions configured to cause the identification tag reader to cooperate with the one or more implantable medical devices and the computing device. (See, for example, system 600 of FIG. 6.) As with the computing device 550, the identification tag reader can include memory (e.g., a non-transitory computer-readable medium) storing the instructions for execution by one or more processors of the identification tag reader configured to cause the identification tag reader to read the identification data for the implantable medical device or write, update, or overwrite the identification data for the implantable medical device. However, such an identification tag reader need not be necessary if the computing device 550, itself, is capable of at least reading electromagnetic identification tags.

FIG. 6 provides a schematic illustrating reading identification data from or writing identification data to an identification tag of the port assembly 100 by the computing device 550 through an intermediate identification tag reader 660 in accordance with some embodiments. As shown, the identification tag reader 660 can be a dedicated RFID tag reader including, but not limited to, Invengo's XC-AT188 RAIN RFID (UHF) handheld reader (Invengo Technology Pte. Ltd., Singapore) in accordance with some embodiments.

Each of the computing device 550 and the identification tag reader 660 can include a short-range wireless-communication interface (e.g., a Bluetooth®), and each of the computing device 550 and the identification tag reader 660 can be further configured via respective instructions thereof to communicate the identification data for the implantable medical device to the other through its short-range wireless-communication interface shown in FIG. 6.

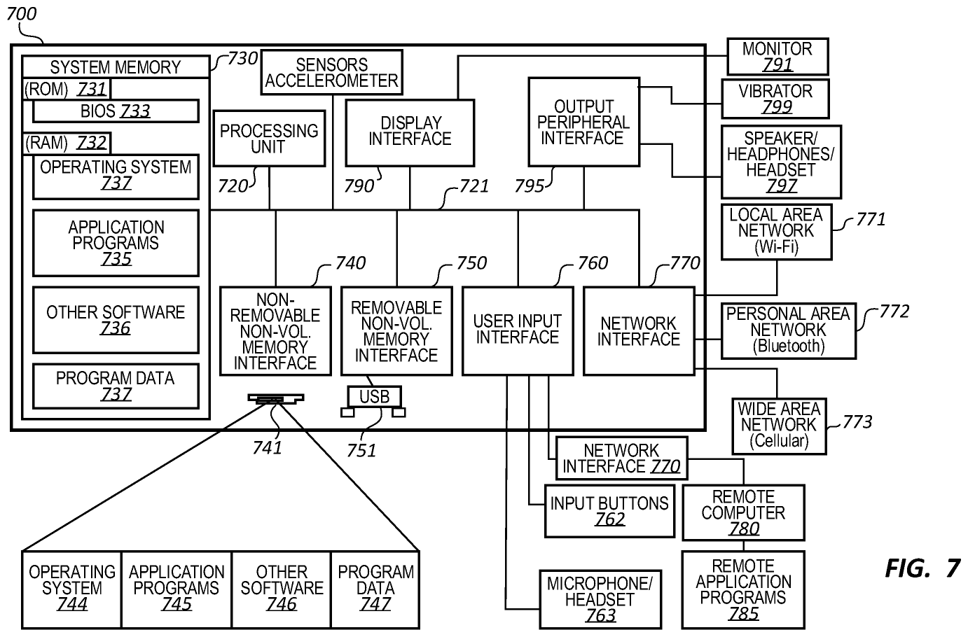

FIG. 7 is a schematic illustrating one or more components of a computing device 700 such as a mobile computing device (e.g., a smartphone, a tablet computer, a dedicated system device, etc.), a wearable computing device (e.g., a smartwatch or an optical head-mounted display, etc.), or an identification tag reader in accordance with some embodiments. The computing device can be partially represented by the one or more components of the computing system 700 or wholly represented by all the components of the computing system 700.

Referring to FIG. 7, components of the computing system 700 can include, but are not limited to, a processing unit 720 having one or more processing cores, a system memory 730, and a system bus 721 that couples various system components including the system memory 730 to the processing unit 720. The system bus 721 can be any of several types of bus structures selected from a memory bus or memory controller, a peripheral bus, and a local bus using any of a variety of bus architectures.

The computing system 700 can include computing machine-readable media. The computing machine-readable media can be any available media that can be accessed by the computing system 700 and includes both volatile and non-volatile media, and removable and non-removable media. By way of example, and not limitation, computing machine-readable media use includes storage of information, such as computer-readable instructions, data structures, other executable software or other data. The computing machine-readable media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible medium that can be used to store the desired information and that can be accessed by the computing device 700. Transitory media such as wireless channels are not included in the computing machine-readable media. Communication media typically embody computer-readable instructions, data structures, other executable software, or other transport mechanisms and include any information delivery media.

The system memory 730 can include computing machine-readable media in the form of volatile and/or non-volatile memory such as read-only memory (ROM) 731 and random access memory (RAM) 732. A basic input/output system 733 (BIOS) containing basic routines configured for transferring information between elements within the computing system 700, such as during start-up, can be stored in the ROM 731. The RAM 732 can contain data and/or software immediately accessible to and/or presently being operated on by the processing unit 720. By way of example, and not limitation, FIG. 7 illustrates that the RAM 732 can include a portion of the operating system 734, the application programs 735, other executable software 736, and the program data 737.

The computing system 700 can also include other removable/non-removable volatile/nonvolatile computing machine-readable media. By way of example only, FIG. 7 illustrates a solid-state memory 741. Other removable/non-removable, volatile/nonvolatile computing machine-readable media that can be used in the example operating environment include, but are not limited to, USB drives and devices, flash memory cards, solid-state RAM, solid-state ROM, and the like. The solid-state memory 741 can be connected to the system bus 721 through a non-removable memory interface such as interface 740, and the USB drive 751 can be connected to the system bus 721 by a removable memory interface, such as interface 750.

The drives and their associated computing machine-readable media discussed above and illustrated in FIG. 7 provide storage of computer-readable instructions, data structures, other executable software and other data for the computing system 700. In FIG. 7, for example, the solid-state memory 741 is illustrated for storing operating system 744, application programs 745, other executable software 746, and program data 747. Note that these components can either be the same as or different from the operating system 734, the application programs 735, the other executable software 736, and the program data 737. The operating system 744, the application programs 745, the other executable software 746, and the program data 747 are given different numbers here to illustrate that, at a minimum, they can be different copies.

A user (e.g., a medical practitioner, etc.) can enter commands and information into the computing system 700 through input devices such as a keyboard, a touchscreen, software or hardware input buttons 762, a microphone 763, or a pointing device or scrolling input component such as a mouse, trackball, or touch pad. The microphone 763 can cooperate with speech recognition software. These and other input devices can be connected to the processing unit 720 through a user interface 760 that is coupled to the system bus 721, but these and other input devices can also be connected by other interface and bus structures such as a parallel port, game port, or USB. A display monitor 791 or other type of display screen device can be connected to the system bus 721 via an interface, such as a display interface 790. In addition to the monitor 791, the computing system 700 can also include other peripheral output devices such as speakers 797, a vibrator 799, and other output devices, which can be connected through an output peripheral interface 795.

The computing system 700 can operate in a networked environment using logical connections to one or more remote computers/client devices, such as a remote computing system 780. The remote computing system 780 can be a server, a personal computer, a hand-held device, a router, a peer device or other common network node, and can include many or all of the elements described above relative to the computing system 700. The logical connections depicted in FIG. 7 can include a personal area network (“PAN”) 772 (e.g., Bluetooth®), a local area network (“LAN”) 771 (e.g., Wi-Fi), and a wide area network (“WAN”) 773 (e.g., cellular network), but the logical connections can also include other networks. Such networking environments can be found in offices, enterprise-wide computer networks, intranets and the Internet. A browser application can be resident on the computing device and stored in the memory.

When used in a LAN networking environment, the computing system 700 can be connected to the LAN 771 through a network interface or adapter 770, which can be, for example, a Wi-Fi adapter. When used in a WAN networking environment (e.g., Internet), the computing system 700 typically includes some means for establishing communications over the WAN 773 such as the network interface 770. With respect to mobile telecommunication technologies, for example, a radio interface, which can be internal or external, can be connected to the system bus 721 via the network interface 770, or some other appropriate mechanism. In a networked environment, other software depicted relative to the computing system 700, or portions thereof, can be stored in a remote memory storage device. By way of example, and not limitation, FIG. 7 illustrates remote application programs 785 as residing on remote computing device 780. It will be appreciated that the network connections shown are examples and other means of establishing a communications link between the computing devices can be used.

In some embodiments, software used to facilitate algorithms discussed herein can be embodied onto a non-transitory machine-readable medium. A machine-readable medium includes any mechanism that stores information in a form readable by a machine (e.g., a computer). For example, a non-transitory machine-readable medium can include read only memory (ROM); random access memory (RAM); magnetic disk storage media; optical storage media; flash memory devices; Digital Versatile Disc (DVD's), EPROMs, EEPROMs, FLASH memory, magnetic or optical cards, or any type of media suitable for storing electronic instructions.

Note, an application described herein includes, but is not limited to, software applications, mobile apps, and programs that are part of an operating system application. Some portions of this disclosure are presented in terms of algorithms and symbolic representations of operations on data bits within a computer memory. These algorithmic descriptions and representations are the means used by those skilled in the data-processing arts to most effectively convey the substance of their work to others skilled in the art. An algorithm is conceived to be a self-consistent sequence of steps leading to a desired result. The steps are those requiring physical manipulations of physical quantities. These quantities can take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. These algorithms can be written in a number of different software programming languages such as C, C+, or other similar languages. Also, an algorithm can be implemented with lines of code in software, configured logic gates in software, or a combination of both. In an embodiment, the logic consists of electronic circuits that follow the rules of Boolean logic, software that contain patterns of instructions, or any combination of both.

It should be borne in mind, however, that all of these and similar terms associated with the appropriate physical quantities are merely convenient labels applied to these quantities. Unless specifically stated otherwise or apparent from the above discussions, it is appreciated that throughout the disclosure terms such as “processing,” “computing,” “calculating,” “determining,” “displaying,” or the like, refer to the action and processes of a computer system (or similar electronic computing system) that manipulates and transforms data represented as physical (electronic) quantities within the computer system's registers and memories into other data similarly represented as physical quantities within the computer system memories or registers, or other such information storage, transmission, or display devices.

Many functions performed by electronic hardware components can be duplicated by software emulation. Thus, a software program written to accomplish those same functions can emulate the functionality of the hardware components in input-output circuitry.

Methods related to implantable medical devices such as port assemblies and systems including such devices include manufacturing the implantable medical devices including embedding at least one UDI such as a machine-readable electromagnetic identification tag in each implantable medical device of the implantable medical devices at a time of manufacture. Alternatively, the at least one UDI can be coupled to each implantable medical device of the implantable medical devices at a time of manufacture. For implantable medical devices having more than one UDI, the UDIs can be embedded, coupled, or both at a time of manufacturing the implantable medical devices. In addition, manufacturing the implantable medical devices can further include packaging the implantable medical devices with UDIs such as machine-readable optical identification tags (e.g., QR codes, UPC barcodes, etc.) also including identification information.

With respect to port assemblies as the implantable medical devices, again, UDIs are counterintuitively embedded in or coupled to catheter locks of the port assemblies despite their diminutive size. In addition to the reasons set forth above, this is also because a UDI such as an RFID tag or NFC tag on a catheter lock can be more easily oriented (e.g., turned) outward during manufacturing for reading identification information during a quality-control check. In addition, such easily read identification information makes it easy to compare the identification information read from the RFID or NFC tag to a QR code or UPC barcode on packaging for the port assembly as an additional quality-control check. That, or the QR code or UPC barcode can be used in a step of writing the identification information to the RFID or NFC tag at a time of manufacturing.

Methods related to implantable medical devices such as port assemblies and systems including such devices include implanting the implantable medical devices. For example, implanting a port assembly includes reading a UDI such as an RFID tag or NFC tag with a computing device or an identification tag reader provided herein for an initial reading to confirm at least the port of the port assembly is the appropriate port and the UDI thereof is in working order; implanting the port of the port assembly in an implant pocket; sliding a catheter over an outlet stem of the port; locking a catheter lock with the RFID tag or NFC tag over both the catheter and the outlet stem; orienting the catheter lock, if needed, to turn the RFID or NFC tag outward from a body of a patient; reading the RFID tag or NFC tag once implanted for a subsequent reading to confirm the UDI is in working order as implanted; suturing the patient; and optionally updating the RFID tag or NFC tag with implant-procedure information and reading it back to confirm.

### Examples

### Complete Device Operation

A Samsung smartphone (Samsung Galaxy S7 smartphone; Samsung Electronics America, Inc.; Ridgefield Park, N.J.) and a Google smartphone (Google Pixel smartphone; Google, LLC; Mountain View, Calif.) were used with three RFID tags (Abracon ART915X0505030P-IC RFID tag; Abracon, LLC; Spicewood, Tex.), three times each, to write and read back identification data by way of a Bluetooth® connection to a handheld RFID reader (Invengo XC-AT188 RAIN RFID (UHF) handheld reader; Invengo Technology Pte. Ltd., Singapore). The identification data used for performance values for the RFID tags included simulated lot numbers, product codes, Groshong line information, MRI standard information, and powered or non-powered indicators. (See, for example, FIG. 5B.) 100% of the tests successfully displayed the correct implantable port information.

### Temperature Test

Nine catheter locks with RFID tags were placed in a furnace at 70° C. for 24 hours, cooled to room temperature, and tested at 37° C. at a minimum acceptable distance to mimic sterilization conditions and real-world use. 100% of the RFID tags were properly read at the end of the test.

### Reading Range

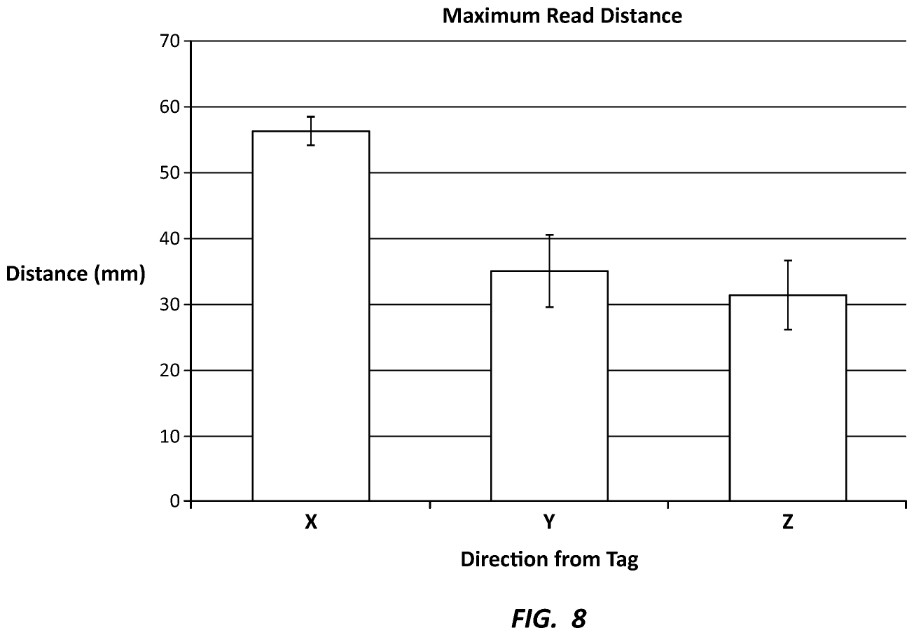

Three RFID tags were tested, three times each, to find a maximum proven reading range. All three RFID tags surpassed a reading range of 25 mm. Table 1 provides results for the reading-range experiment. FIG. 8 is an example reading-range chart providing reading-range data for an experimental run for another RFID tag that provided data similar to Tag 3, Sample #8 from Table 1.

TABLE 1
Results from the reading-range experiment.

| Tag 1 | | Tag 2 | | Tag 3 | |
|---|---|---|---|---|---|
| Run # | Result | Run # | Result | Run # | Result |
| 1 | | 4 | | 7 | |
| Max X | 57 mm | Max X | 60 mm | Max X | 57 mm |
| Max Y | 40 mm | Max Y | 42 mm | Max Y | 34 mm |
| Max H | 28 mm | Max H | 40.5 mm | Max H | 35.5 mm |
| 2 | | 5 | | 8 | |
| Max X | 57 mm | Max X | 60 mm | Max X | 57 mm |
| Max Y | 40 mm | Max Y | 42 mm | Max Y | 35 mm |
| Max H | 30 mm | Max H | 40.5 mm | Max H | 35.5 mm |
| 3 | | 6 | | 9 | |
| Max X | 55 mm | Max X | 60 mm | Max X | 57 mm |
| Max Y | 40 mm | Max Y | 42 mm | Max Y | 34 mm |
| Max H | 30 mm | Max H | 40.5 mm | Max H | 35.5 mm |

| | |
|---|---|
| Performance | Mobile application displays correct tag |
| values: | information from greater than 25 mm but less |
| | than 1 m from an RFID tag. |

Reading Range: PermaGel Skin Substitute

Three RFID tags embedded in respective catheter locks were tested, three times each, under conditions mimicking the real-world conditions in which implantable medical devices such as the implantable ports and power injectable ports provided herein will be used. In order to mimic the real-world conditions, a 25-mm thick piece of PermaGel skin substitute (10% Ballistic Gelatin Air Rifle Block; Clear Ballistics LLC; Fort Smith, Ark.) was placed over each RFID tag-containing catheter lock for reading the RFID tags. For each run, the RFID tag was read at a distance between 0 and 5 mm above the PermaGel skin substitute. All three RFID tags surpassed a reading range of 25 mm through the PermaGel skin substitute as well as an additional 0-5 mm of air above the PermaGel skin substitute.

### Tensile Strength

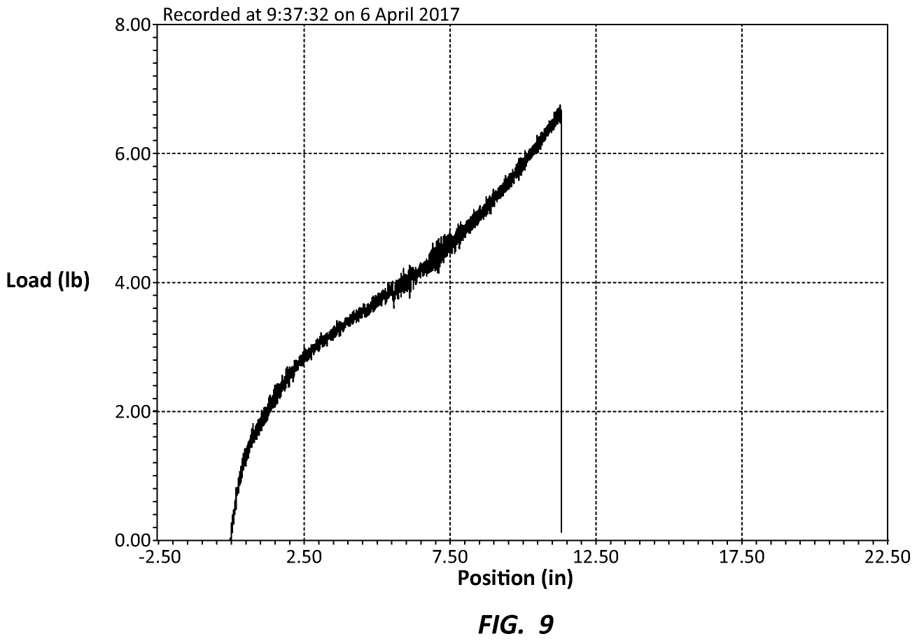

Three catheter-lock prototypes were tested for tensile strength using a validated universal testing machine. The catheter-lock prototypes were preconditioned in an environment of 100% humidity and subsequently pulled at 20 inch/min until failure. All three catheter-lock prototypes surpassed the requirement of withstanding 15N by at least a 33% margin. Table 2 provides results for the tensile-strength experiment. FIG. 9 is a tensile-strength chart providing tensile-strength data involving Prototype 3, Run #8 of Table 2. Additional tensile-strength charts for the other tensile strength runs can be found in U.S. Provisional Patent Application No. 62/491,846, filed Apr. 28, 2017, which is hereby incorporated herein by reference in its entirety.

TABLE 2
Results from the tensile-strength experiment.

| Prototype 1 | | Prototype 2 | | Prototype 3 | |
|---|---|---|---|---|---|
| Run # | Result | Run # | Result | Run # | Result |
| 1 | 31.026 N | 4 | 24.158 N | 7 | 30.030 N |
| | Pass | | Pass | | Pass |
| 2 | 21.752 N | 5 | 31.600 N | 8 | 27.076 N |
| | Pass | | Pass | | Pass |
| 3 | 31.026 N | 6 | 23.709 N | 9 | 20.907 N |
| | Pass | | Pass | | Pass |

| | |
|---|---|
| Performance | Device does not break under 15 N of force. |
| values: | |

### Time Response

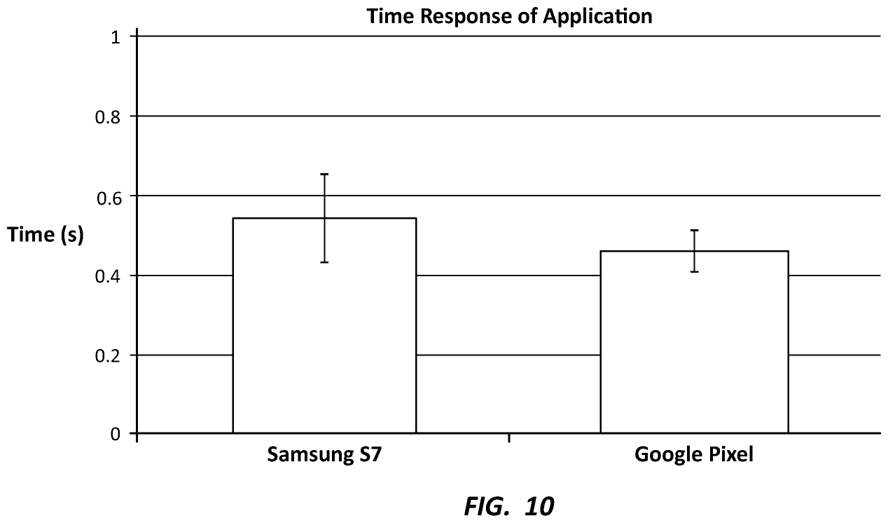

Three RFID tags were tested, 10 times each, for each smartphone of the Samsung Galaxy S7 and Google Pixel smartphones. Data was taken at the maximum proven communication range found during the reading-range experiment from the point of pressing “read” in the GUI-based application (see GUI of FIG. 5A) until results were displayed. Table 3 and 4 provide results for the time-response experiment. FIG. 10 is a time-response chart comparing the response times of the Samsung Galaxy S7 and Google Pixel smartphones using the time-response data for the time-response experiment from Tables 3 and 4.

TABLE 3
Results from the time-response experiment for
Samsung Galaxy S7 smartphone.

| Tag 1 | | Tag 2 | | Tag 3 | |
|---|---|---|---|---|---|
| Run # | Result | Run # | Result | Run # | Result |
| 1 | 0.89 sec | 11 | 0.73 sec | 21 | 0.61 sec |
| | pass | | Pass | | pass |
| 2 | 0.48 sec | 12 | 0.60 sec | 22 | 0.51 sec |
| | pass | | Pass | | pass |
| 3 | 0.55 sec | 13 | 0.43 sec | 23 | 0.53 sec |
| | pass | | Pass | | pass |
| 4 | 0.76 sec | 14 | 0.48 sec | 24 | 0.55 sec |
| | pass | | Pass | | pass |
| 5 | 0.56 sec | 15 | 0.52 sec | 25 | 0.36 sec |
| | pass | | Pass | | pass |
| 6 | 0.63 sec | 16 | 0.55 sec | 26 | 0.51 sec |
| | pass | | Pass | | pass |
| 7 | 0.49 sec | 17 | 0.58 sec | 27 | 0.55 sec |
| | pass | | Pass | | pass |
| 8 | 0.45 sec | 18 | 0.45 sec | 28 | 0.43 sec |
| | pass | | Pass | | pass |
| 9 | 0.51 sec | 19 | 0.53 sec | 29 | 0.60 sec |
| | Pass | | Pass | | pass |
| 10 | 0.53 sec | 20 | 0.58 sec | 30 | 0.51 sec |
| | pass | | Pass | | pass |

| | |
|---|---|
| Performance | Samsung Galaxy S7 smartphone displays correct tag |
| values: | contents within 5 seconds of entering the maximum |
| | proven communication range found during the |
| | reading-range experiment. |

TABLE 4
Results from the time-response experiment for
Google Pixel smartphone.

| Tag 1 | | Tag 2 | | Tag 3 | |
|---|---|---|---|---|---|
| Run # | Result | Run # | Result | Run # | Result |
| 1 | 0.48 sec | 11 | 0.53 sec | 21 | 0.51 sec |
| | pass | | Pass | | pass |
| 2 | 0.38 sec | 12 | 0.48 sec | 22 | 0.41 sec |
| | pass | | Pass | | pass |
| 3 | 0.44 sec | 13 | 0.53 sec | 23 | 0.48 sec |
| | pass | | Pass | | pass |
| 4 | 0.41 sec | 14 | 0.45 sec | 24 | 0.51 sec |
| | pass | | Pass | | pass |
| 5 | 0.43 sec | 15 | 0.43 sec | 25 | 0.41 sec |
| | pass | | Pass | | pass |
| 6 | 0.48 sec | 16 | 0.43 sec | 26 | 0.45 sec |
| | pass | | Pass | | pass |
| 7 | 0.53 sec | 17 | 0.46 sec | 27 | 0.58 sec |
| | pass | | Pass | | pass |
| 8 | 0.43 sec | 18 | 0.43 sec | 28 | 0.41 sec |
| | pass | | Pass | | pass |
| 9 | 0.46 sec | 19 | 0.55 sec | 29 | 0.41 sec |
| | pass | | Pass | | pass |
| 10 | 0.53 sec | 20 | 0.46 sec | 30 | 0.40 sec |
| | pass | | Pass | | pass |

| | |
|---|---|
| Performance | Google Pixel smartphone displays correct tag |
| values: | contents within 5 seconds of entering the maximum |
| | proven communication range found during the |
| | reading-range experiment. |

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures can be made from the particular embodiments disclosed herein without departing from the scope of the concepts provided herein.

What is claimed is:

1. An implantable medical device, comprising:
a catheter lock configured to fit over an end portion of a catheter over an outlet stem extending from a portion of the implantable medical device; and
one or more unique device identifiers (“UDIs”) embedded in the catheter lock, the one or more UDIs including machine-readable identification data for the implantable medical device.

2. The implantable medical device of claim 1, wherein the identification data for the implantable medical device is identification data for a port assembly.

3. The implantable medical device of claim 1, wherein:
two or more UDIs are embedded in the catheter lock approximately equally spaced around the catheter lock, and
each UDI of the two or more UDIs includes the same identification data for the implantable medical device.

4. The implantable medical device of claim 1, wherein each UDI is an identification tag selected from a radio-frequency identification (“RFID”) tag and a near-field communication (“NFC”) tag.

5. The implantable medical device of claim 4, wherein each UDI is an RFID tag.

6. The implantable medical device of claim 4, wherein each UDI is a passive RFID tag.

7. A system, comprising:
an implantable medical device, comprising:
a catheter lock configured to fit over an end portion of a catheter over an outlet stem extending from a portion of the implantable medical device; and
one or more unique device identifiers (“UDIs”) embedded in or coupled to the catheter lock, the one or more UDIs including machine-readable identification data for the implantable medical device; and
instructions stored in a memory of a computing device for execution by one or more processors of the computing device configured to cause the computing device to present the identification data for the implantable medical device to a user on a display screen associated with the computing device.

8. The system of claim 7, wherein:
the implantable medical device includes a port assembly, and
the identification data for the implantable medical device includes identification data for the port assembly selected from maker of a port of the port assembly, model of the port, lot number for a lot of the port, serial number of the port, magnetic resonance imaging (“MRI”) safety information for the port or the port assembly, and a description of the port assembly.

9. The system of claim 8, wherein:
the port includes a power injectable port configured for mechanically assisted pressurized injections to achieve a desired flow rate of injectant through the port assembly.

10. The system of claim 7, wherein each UDI is an identification tag selected from a radio-frequency identification (“RFID”) tag and a near-field communication (“NFC”) tag.

11. The system of claim 10, wherein each UDI is an RFID tag.

12. The system of claim 10, wherein each UDI is an NFC tag.

13. The system of claim 10, wherein the instructions are further configured to cause the computing device to accept user input through a user-input mechanism of the computing device for updating or overwriting the identification data for the implantable medical device in each UDI.

14. The system of claim 13, further comprising a dedicated UDI reader including memory storing instructions for execution by one or more processors of the UDI reader configured to cause the UDI reader to read the identification data for the implantable medical device and optionally update or overwrite the identification data for the implantable medical device in each UDI.

15. The system of claim 15, wherein the computing device and the UDI reader are each further configured via respective instructions thereof to communicate the identification data for the implantable medical device to the other through a short-range wireless-communication interface.

16. The system of claim 7, wherein the instructions are configured for a computing device selected from:
i) a mobile computing device including a smartphone, a tablet computer, and a dedicated system device, and
ii) a wearable computing device including a smartwatch and an optical head-mounted display.

17. A non-transitory computer-readable medium including instructions for execution by one or more processors of a computing device configured to cause the computing device to perform operations, comprising:
presenting identification data to a user in one or more graphical user interfaces on a display screen associated with the computing device read from one or more unique device identifiers (“UDIs”) of a port assembly,
wherein the port assembly includes a catheter lock configured to fit over an end portion of a catheter over an outlet stem extending from a housing of a port, the one or more UDIs are embedded in or coupled to the catheter lock, and each UDI of the one or more UDIs is an identification tag selected from a radio-frequency identification (“RFID”) tag and a near-field communication (“NFC”) tag.

18. The computer-readable medium of claim 17, wherein the instructions are further configured to cause the computing device to accept user input through a user-input mechanism of the computing device for updating or overwriting the identification data for the port assembly in each UDI.

19. The computer-readable medium of either claim 17, wherein:
each UDI of the one or more UDIs is an RFID tag, and
the instructions are further configured to cause the computing device to cooperate with an RFID tag reader over a short-range wireless-communication interface of the computing device for communications regarding the identification data.

20. The system of claim 17, wherein the port is a power injectable port configured for mechanically assisted pressurized injections to achieve a desired flow rate of injectant through the port assembly.
